SEQUENCE LISTING (17 sequences)

SEQ ID NO: 1 - protein, 353 aa, Artificial Sequence
  MCH-1R antagonist binding protein
MDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDHYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 2 - protein, 353 aa, Artificial Sequence
  MCH-1R antagonist binding protein
MDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFAKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 3 - protein, 353 aa, Artificial Sequence
  MCH-1R antagonist binding protein
MDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYFTIFYALQYHNIMTVKRATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 4 - protein, 567 aa, Artificial Sequence
  MCH-1R antagonist binding protein
MDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCEVDGTAGPGSIATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - DNA, 1065 bp, Artificial Sequence
  MCH-1R antagonist binding protein
atggacctggaagcctcgctgctgcccactggtcccaatgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccactacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcacctgatac

SEQ ID NO: 6 - DNA, 1065 bp, Artificial Sequence
  MCH-1R antagonist binding protein
atggacctggaagcctcgctgctgcccactggtcccaatgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttcgcgaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcacctgatac

SEQ ID NO: 7 - DNA, 1065 bp, Artificial Sequence
  MCH-1R antagonist binding protein
atggacctggaagcctcgctgctgcccactggtcccaatgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctactttactatcttctatgctctccagtaccataacattatgacagttaagcgggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcacctgatac

SEQ ID NO: 8 - DNA, 1704 bp, Artificial Sequence
  MCH-1R antagonist binding protein
atggacctggaagcctcgctgctgcccactggtcccaatgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgaggtcgacggtaccgcgggcccgggatccatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - DNA, 33 bp, Artificial Sequence
  Oligonucleotide primer
ccatggccattgaccactacctggccactgtcc

SEQ ID NO: 10 - DNA, 33 bp, Artificial Sequence
  Oligonucleotide primer
ggacagtggccaggtagtggtcaatggccatgg

SEQ ID NO: 11 - DNA, 34 bp, Artificial Sequence
  Oligonucleotide primer
ctcttccacgaagttcgcgaagccctctgtggcc

SEQ ID NO: 12 - DNA, 34 bp, Artificial Sequence
  Oligonucleotide primer
ggccacagagggcttcgcgaacttcgtggaagag

SEQ ID NO: 13 - DNA, 47 bp, Artificial Sequence
  Oligonucleotide primer
tttgtgtacatcgtgctctgtgaggtcgacggtaccgcgggcccggg

SEQ ID NO: 14 - DNA, 47 bp, Artificial Sequence
  Oligonucleotide primer
cccgggcccgcggtaccgtcgacctcacagagcacgatgtacacaaa

SEQ ID NO: 15 - protein, 27 aa, Artificial Sequence
  Region between TM3 and TM4 in MCH-1R(i2/MC4R)
DRYFTIFYALQYHNIMTVKRATLVICL

SEQ ID NO: 16 - protein, 11 aa, Artificial Sequence
  MCH-1R antagonist
XCMLGRVYXXC

SEQ ID NO: 17 - protein, 11 aa, Artificial Sequence
  MCH-1R antagonist
XCMLXRVYXXC

(19) United States
(12) Patent Application Publication
(10) Pub. No.: US 2005/0069883 A1
(43) Pub. Date: Mar. 31, 2005

(54) Melanin-concentrating hormone receptor antagonist binding protein

(76) Inventors: Andrew D. Howard, Park Ridge, NJ (US); Andreas W. Sailer, Edison, NJ (US); Jie Pan, Plainsboro, NJ (US); Tung M. Fong, Somerset, NJ (US); Donald J. Marsh, Hillsborough, NJ (US)

Correspondence Address: MERCK AND CO., INC, P O BOX 2000, RAHWAY, NJ, 07065-0907, US

(21) Appl. No.: 10/488,758

(22) PCT Filed: Sep. 20, 2002
(86) PCT No.: PCT/US02/29931

Related U.S. Application Data
(60) Provisional application No. 60/325,129, filed on Sep. 26, 2001.

(51) Int. Cl. A61K 38/24; C12Q 1/68; C07H 21/04; C07K 14/575

(52) U.S. Cl. 435/6; 435/69.1; 435/320.1; 435/325; 514/12; 530/399; 536/23.5

(57) ABSTRACT

The present invention features MCH-1R antagonist binding proteins. MCH-1R antagonist binding proteins described herein are based on an MCH-1R having one or more alterations to the second intracellular loop or carboxy terminus that render the receptor substantially inactive to MCH binding. An MCH-1R antagonist binding protein can bind MCH-1R antagonists, but does not exhibit high affinity MCH binding and is not activated by the MCH.

### CROSS-REFERENCE TO RELATED APPLICATIONS

The present application claims priority to provisional application U.S. Ser. No. 60/325,129, filed Sep. 26, 2001, hereby incorporated by reference herein.

### BACKGROUND OF THE INVENTION

The references cited in the present application are not admitted to be prior art to the claimed invention.

Neuropeptides present in the hypothalamus play a major role in mediating the control of body weight. (Flier, et al., 1998. Cell, 92, 437-440.) Melanin-concentrating hormone (MCH) is a cyclic 19-amino acid neuropeptide synthesized as part of a larger pre-prohormone precursor in the hypothalamus which also encodes neuropeptides NEI and NGE. (Nahon, et al., 1990. Mol. Endocrinol. 4, 632-637.) MCH was first identified in salmon pituitary, and in fish MCH affects melanin aggregation thus affecting skin pigmentation. In trout and in eels MCH has also been shown to be involved in stress induced or CRF-stimulated ACTH release. (Kawauchi, et al., 1983. Nature 305, 321-323.)

In humans two genes encoding MCH have been identified that are expressed in the brain. (Breton, et al., 1993. Mol. Brain Res. 18, 297-310.) In mammals MCH has been localized primarily to neuronal cell bodies of the hypothalamus which are implicated in the control of food intake, including perikarya of the lateral hypothalamus and zona inertia. (Knigge, et al., 1996. Peptides 17, 1063-1073.)

Pharmacological and genetic evidence suggest that the primary mode of MCH action is to promote feeding (orexigenic). MCH mRNA is up regulated in fasted mice and rats and in the ob/ob mouse. (Qu, et al., 1996. Nature 380, 243-247.) Injection of MCH centrally (ICV) stimulates food intake and MCH antagonizes the hypophagic effects seen with α melanocyte stimulating hormone (αMSH). (Qu, et al., 1996. Nature 380, 243-247.) MCH deficient mice are lean, hypophagic and have increased metabolic rate. (Shimada, et al., 1998. Nature 396, 670-673.) Transgenic mice overexpressing MCH are hyperphagic and develop insulin resistance and mild obesity. (Ludwig, et al., 2001, J. Clin. Invest. 107, 379-386.)

MCH action is not limited to modulation of food intake as effects on the hypothalamic-pituitary-axis have been reported. (Nahon, 1994. Critical Rev. in Neurobiol. 8, 221-262.) MCH can modulate stress-induced release of ACTH. (Nahon, 1994. Critical Rev. in Neurobiol. 8, 221-262.)

Several references describe a human melanin-concentrating hormone receptor (“MCH-1R”). (Chambers, et al., 1999. Nature 400, 261-265, Saito, et al., 1999. Nature 400, 265-269, Bächner, et al., 1999. FEBS Letters 457:522-524, Shimomura, et al., 1999. Biochemical and Biophysical Research Communications 261, 622-626.)

### SUMMARY OF THE INVENTION

The present invention features MCH-1R antagonist binding proteins. MCH-1R antagonist binding proteins described herein are based on an MCH-1R having one or more alterations to the second intracellular loop or carboxy terminus that render the receptor substantially inactive to MCH binding. An MCH-1R antagonist binding protein can bind MCH-1R antagonists, but does not exhibit high affinity MCH binding and is not activated by the MCH.

Thus, a first aspect of the present invention describes an MCH-1R antagonist binding protein selected from the group consisting of:
a) a MCH-1R antagonist binding protein having one or more alterations in the second intracelullar loop region that render MCH-1R substantially inactive to MCH binding; and
b) a MCH-1R antagonist binding protein having one or more alterations in the C-terminal that render MCH-1R substantially inactive to MCH binding.

“Substantially inactive to MCH binding” indicates that MCH binding, if present, is up to about 10% the level of binding to human MCH-1R. In different embodiments binding is 5% or less, and undetectable.

Another aspect of the present invention describes a nucleic acid comprising a nucleotide sequence encoding an MCH-1R antagonist binding protein. In an embodiment of the present invention, the nucleic acid is an expression vector.

Another aspect of the present invention describes a recombinant cell comprising an expression vector encoding an MCH-1R antagonist binding protein. The nucleotide sequence encoding the MCH-1R antagonist binding protein is functionally coupled to a promoter recognized by the cell.

Another aspect of the present invention describes a method of screening for a compound able to bind an MCH-1R antagonist binding protein. The method involves contacting an MCH-1R antagonist binding protein with the compound and measuring the ability of the compound to bind to the protein.

Another aspect of the present invention describes a method of preparing a MCH-1R antagonist binding protein. The method involves growing a recombinant cell containing an expression vector encoding an MCH-1R antagonist binding protein.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 illustrates [¹²⁵I]Phe¹³Tyr¹⁹-MCH binding to MCH-1R(R141H) and MCH-1R(R155A).

FIG. 2 illustrates [¹²⁵I]Phe¹³Tyr¹⁹-MCH binding to MCH-1R(Δ316/EGFP).

FIG. 3 illustrates the lack of functional activation of MCH-1R(R141H) by MCH. Functional activation was assayed by measuring mobilization of intracellular calcium.

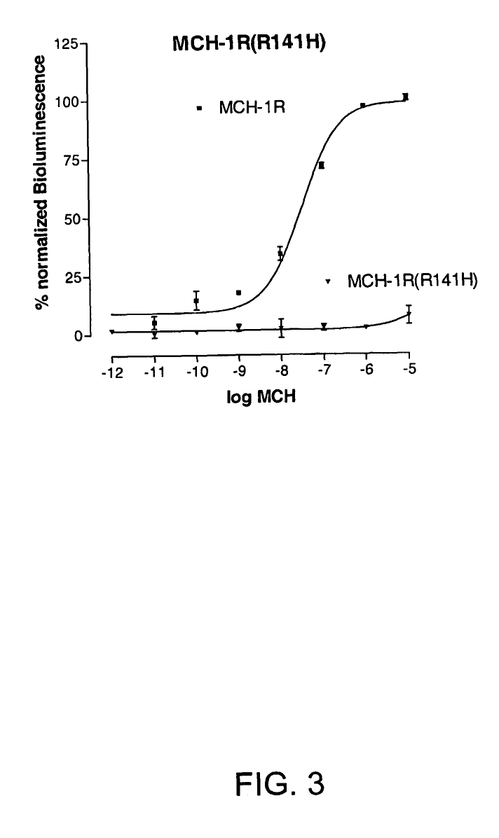

FIG. 4 illustrates the lack of functional activation of MCH-1R(R155A) by MCH. Functional activation was assayed by measuring mobilization of intracellular calcium.

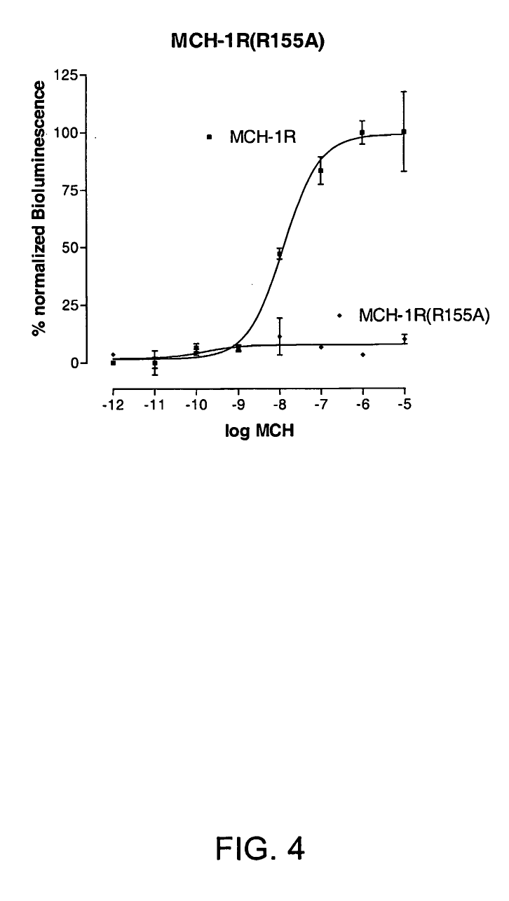

FIG. 5 illustrates the lack of functional activation of MCH-1R(i2MC4R) by MCH. Functional activation was assayed by measuring mobilization of intracellular calcium.

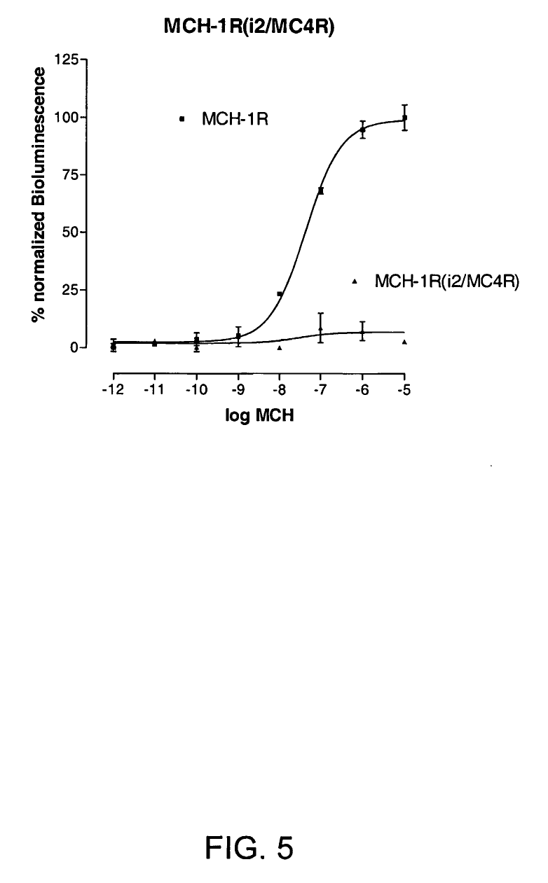

FIG. 6 illustrates the lack of functional activation of MCH-1R(Δ316/EGFP) by MCH. Functional activation was assayed by measuring mobilization of intracellular calcium.

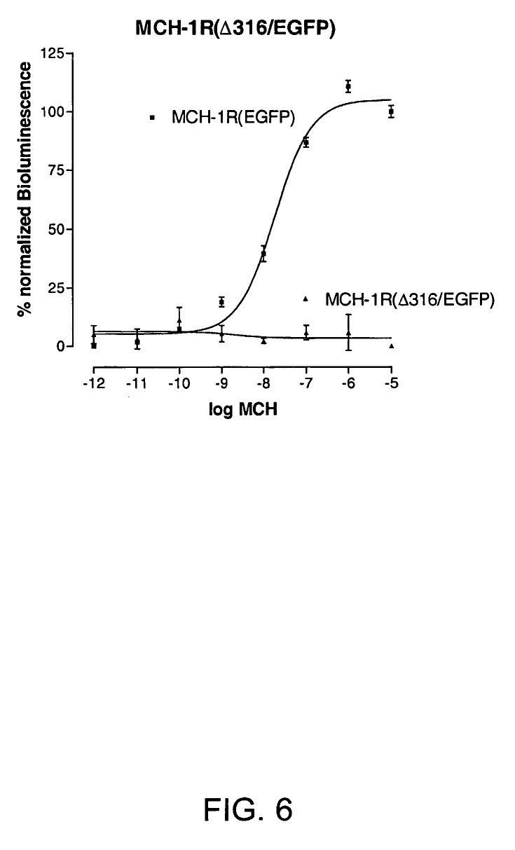

### DETAILED DESCRIPTION OF THE INVENTION

Directed mutagenesis of the human MCH-1R has generated MCH-1R antagonist binding proteins that selectively bind MCH-1R antagonists. The MCH-1R antagonists do not exhibit high affinity MCH agonist binding and are not activated by MCH. Uses of MCH-1R antagonist binding proteins include screening for potential receptor antagonists and studying protein trafficking.

Different types of MCH-1R antagonist binding proteins were obtained by altering MCH-1R in the second intracellular loop region and by deleting a portion of the carboxy terminus. Alterations to the second intracellular loop region to produce a MCH-1R antagonist binding protein include single and multiple amino acid changes.

The MCH-1R antagonist binding proteins MCH-1R(R141H) and MCH-1R(R155A) contain single amino acid changes in the second intracellular loop region of MCH-1R. The amino acid sequences of MCH-1R(R141H) and MCH-1R(R155A) are provided by SEQ. ID. NO. 1 and SEQ. ID. NO. 2.

Position 141 is within the highly conserved DRY signature sequence found in most G-protein coupled receptors. The DRY signature sequence has been suggested to be involved in G-protein interaction. (Rosenthal, et al., J. Biol. Chem. 268:13030-3, 1993.)

The MCH-1R antagonist binding protein MCH-1R(i2/MC4R) contains the MCH-1R, except the second intracellular loop which is replaced by the corresponding second intracellular loop of human MC4R. The amino acid sequence of MCH-1R(i2/MC4R) is provided by SEQ. ID. NO. 3.

MC4R is the melanocortin-4 receptor. (Yang et al., Biochemistry 39: 14900-11, 2000, Gantz et al., J. Biol. Chem. 268:15174-9, 1993.) Alterations to MC4R are described, for example, by Fraendberg, et al. Biochem. Biophys. Res. Commun. 245:490-492, 1998.

An example of a C-terminal deletion is provided by MCH-1R(Δ316/EGFP) where the C-terminal 37 amino acids of MCH-1R was deleted and the enhanced green fluorescence protein (EGFP) was added to the C-terminus. C-terminal deletions to the human somatostatin receptor type 5 have been described by Hukovic, et al. Journal of Biological Chemistry 273:21416-21422, 1998.

The amino acid sequence of MCH-1R(Δ316/EGFP) is provided by SEQ. ID. NO. 4. The EGFP sequence facilitates the study of protein trafficking.

Production of MCH-1R Antagonist Binding Protein

Different MCH-1R antagonist binding protein can be obtained based on the guidance provided herein. The provided guidance includes the identification of particular mutations and regions useful for producing MCH-1R binding antagonists. Preferred MCH-1R antagonist binding proteins are based on the human MCH-1R sequence.

MCH-1R antagonist binding protein should be able to bind an MCH antagonist, but not MCH. Different MCH-1R antagonist binding proteins can be produced, for example, by starting with an MCH-1R antagonist binding protein described herein and making additional alterations.

Alterations to a polypeptide not expected to alter polypeptide functioning can be made taking into account amino acid R groups. Differences in naturally occurring amino acids are due to different R groups. An R group affects different properties of an amino acid such as physical size, charge, and hydrophobicity. Amino acids can be divided into different groups as follows: neutral and hydrophobic (alanine, valine, leucine, isoleucine, proline, tryptophan, phenylalanine, and methionine); neutral and polar (glycine, serine, threonine, tyrosine, cysteine, asparagine, and glutamine); basic (lysine, arginine, and histidine); and acidic (aspartic acid and glutamic acid).

Generally, in substituting different amino acids to maintain antagonist binding it is preferable to exchange amino acids having similar properties. Substituting different amino acids within a particular group, such as substituting valine for leucine, arginine for lysine, and asparagine for glutamine are good candidates for not causing a change in antagonist binding.

In different embodiments MCH-1R antagonist binding protein: (1) have a sequence similarity of at least about 90%, preferably at least about 95% with either SEQ. ID. NOs. 1, 2, 3, 4, or a human MCH-1R with a deletion of about 37 amino acids; or (2) provide a sequence with up to about 20 alterations from SEQ. ID. NOs. 1, 2, 3, 4, or a human MCH-1R with a deletion of about 37 amino acids. Sequence similarity for polypeptides can be determined by the BLAST. (Altschul, et al., 1997. Nucleic Acids Res. 25, 3389-3402, hereby incorporated by reference herein.) In one embodiment sequence similarity is determined using tBLASTn search program with the following parameters: MATRIX:BLOSUM62, PER RESIDUE GAP COST: 11, and Lambda ratio: 1.

Alterations to amino acid sequences are additions, deletions, and substitutions. In different embodiments the MCH-1R polypeptide has 1, 2, 3, 4, 5, 6, 7, 8, 9, 10, or 10-20, alterations from SEQ. ID. NOs. 1, 2, 3, 4, or a human MCH-1R with a deletion of about 37 amino acids.

MCH-1R antagonist binding protein can be synthesized using standard techniques including those involving chemical synthesis and those involving biochemical synthesis. Techniques for chemical synthesis of polypeptides are well known in the art. (See e.g., Vincent, in Peptide and Protein Drug Delivery, New York, N.Y., Dekker, 1990.)

Biochemical synthesis techniques for polypeptides are also well known in the art. Such techniques employ a nucleic acid template for polypeptide synthesis. Examples of techniques for introducing nucleic acid into a cell and expressing the nucleic acid to produce protein are provided in references such as Ausubel, Current Protocols in Molecular Biology, John Wiley, 1987-1998, and Sambrook, et al., Molecular Cloning, A Laboratory Manual, 2^{nd} Edition, Cold Spring Harbor Laboratory Press, 1989.

Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. Amino acids are encoded by codons as follows:

A = Ala = Alanine:
codons GCA, GCC, GCG, GCU
C = Cys = Cysteine:
codons UGC, UGU
D = Asp = Aspartic acid:
codons GAC, GAU
E = Glu = Glutamic acid:
codons GAA, GAG
F = Phe = Phenylalanine:
codons UUC, UUU
G = Gly = Glycine:
codons GGA, GGC, GGG, GGU
H = His = Histidine:
codons CAC, CAU
I = IIe = Isoleucine:
codons AUA, AUC, AUU
K = Lys = Lysine:
codons AAA, AAG
L = Leu = Leucine:
codons UUA, UUG, CUA, CUC, CUG, CUU
M = Met = Methionine:
codon AUG
N = Asn = Asparagine:
codons AAC, AAU
P = Pro = Proline:
codons CCA, CCC, CCG, CCU
Q = Gln = Glutamine:
codons CAA, CAG
R = Arg = Arginine:
codons AGA, AGG, CGA, CGC, CGG, CGU
S = Ser = Serine:
codons AGC, AGU, UCA, UCC, UCG, UCU
T = Thr = Threonine:
codons ACA, ACC, ACG, ACU
V = Val = Valine:
codons GUA, GUC, GUG, GUU
W = Trp = Tryptophan:
codon UGG
Y = Tyr = Tyrosine:
codons UAC, UAU

Examples of nucleotide sequences encoding MCH-1R antagonist binding protein based on the human MCH-1R are provided by:
SEQ. ID. NO. 5: nucleotide MCH-1R(R141H);
SEQ. ID. NO. 6: nucleotide MCH-1R(R155A);
SEQ. ID. NO. 7: nucleotide MCH-1R(i2/MC4R); and
SEQ. ID. NO. 8: nucleotide MCH-1R(Δ316/EGFP).

In different embodiments nucleic acid encoding a MCH-1R antagonist binding protein: (1) encode a protein having a sequence similarity of at least about 90%, preferably at least about 95% with either SEQ. ID. NOs. 1, 2, 3, 4, or a human MCH-1R with a deletion of about 37 amino acids; (2) encode a protein having a sequence with up to about 20 alterations from SEQ. ID. NOs. 1, 2, 3, 4, or a human MCH-1R with a deletion of about 37 amino acids; (3) the nucleic acid has a sequence similarity of at least about 90%, or at least about 95% with SEQ. ID. NO. 5, 6, 7, 8, or the human MCH-1R nucleic acid sequence with a deletion corresponding to about 37 C-terminal amino acids.

Sequence similarity for nucleic acid can be determined by FASTA. (Pearson 1990. Methods in Enzymology 183, 63-98, hereby incorporated by reference herein.) In one embodiment, sequence similarity is determined using the FASTA search program with the following parameters: MATRIX: BLOSUM50, GAP PENALTIES: open=−12; residue=−2.

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., Molecular Cloning, A Laboratory Manual, 2^{nd} Edition, Cold Spring Harbor Laboratory Press, 1989.

Biochemical nucleic acid synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and Kacian, et al., U.S. Pat. No. 5,480,784.

In an embodiment of the present invention, the MCH-1R antagonist binding protein is a purified polypeptide. A “purified polypeptide” represents at least 10% of the total protein present in a sample or preparation. In additional embodiments, the purified polypeptide represents at least about 50%, at least about 75%, or at least about 95% of the total protein in a sample or preparation. Reference to “purified polypeptide” does not require that the polypeptide has undergone any purification and may include, for example, chemically synthesized polypeptide that has not undergone any purification steps.

Recombinant Expression

MCH-1R antagonist binding protein can be expressed from recombinant nucleic acid in a suitable host or in a test tube using a translation system. Recombinantly expressed MCH-1R antagonist binding protein are preferably used in assays to screen for compounds that bind to MCH-1R and modulate MCH-1R activity.

Preferably, expression is achieved in a host cell using an expression vector. An expression vector is made up of recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Expression vectors providing suitable levels of polypeptide expression in different hosts are well known in the art. Mammalian expression vectors well known in the art include pcDNA3 (Invitrogen), pMC1neo (Stratagene), pXT1 (Stratagene), pSG5 (Stratagene), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), pCI-neo (Promega) and .lambda.ZD35 (ATCC 37565). Bacterial expression vectors well known in the art include pET11a (Novagen), lambda gt11 (Invitrogen), pcDNAII (Invitrogen), and pKK223-3 (Pharmacia). Fungal cell expression vectors well known in the art include pYES2 (Invitrogen) and Pichia expression vector (Invitrogen). Insect cell expression vectors well known in the art include Blue Bac III (Invitrogen).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK.sup.-) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) and MRC-5 (ATCC CCL 171).

To enhance expression in a particular host it may be useful, for example, to modify the sequence provided in SEQ. ID. NOs. 5, 6, 7, or 8 to take into account codon usage of the host. Codon usage of different organisms are well known in the art. (See, Ausubel, Current Protocols in Molecular Biology, John Wiley, 1987-1998, Supplement 33 Appendix IC.)

Expression vectors may be introduced into host cells using standard techniques. Examples of such techniques include transformation, transfection, lipofection, protoplast fusion, and electroporation.

Nucleic acid encoding an MCH-1R antagonist binding protein can be expressed in a cell without using of an expression vector by, for example, introducing a recombinant nucleic acid encoding the protein into the cell genome. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection.

Functional Assays

Evaluating the ability of a potential MCH-1R antagonist to modulate MCH-1R activity is facilitated through the use of an assay involving a functional MCH-1R and an MCH agonist. The use of an MCH agonist provides for MCH-1R activity.

Recombinantly expressed MCH-1R can be used to facilitate determining receptor activity. For example, MCH-1R can be expressed by an expression vector in a cell line such as HEK 293, COS 7, or CHO, not normally expressing the receptor, wherein the same cell line without the expression vector or with an expression vector not encoding MCH-1R can act as a control.

Functional assays can be performed using individual compounds or preparations containing different compounds. A preparation containing different compounds where one or more compounds affect MCH-1R activity can be divided into smaller groups of compounds to identify the compound(s) affecting MCH-1R activity.

Modulating MCH-1R Activity

MCH-1R antagonists have a variety of different uses including utility as a tool to further study MCH-1R activity and as an agent to achieve a beneficial effect in a patient. Beneficial effects of an MCH-1R antagonist include achieving one or more of the following in a patient: weight loss, cancer treatment (e.g., colon or breast), pain reduction, diabetes treatment, stress reduction and sexual dysfunction treatment.

A patient is a mammal, preferably a human. Reference to patient does not necessarily indicate the presence of a disease or disorder. The term patient includes subjects treated prophylactically and subjects afflicted with a disease or disorder.

Excessive weight is a contributing factor to different diseases including hypertension, diabetes, dyslipidemias, cardiovascular disease, gall stones, osteoarthritis and certain forms of cancers. Bringing about a weight loss can be used, for example, to reduce the likelihood of such diseases and as part of a treatment for such diseases. Weight reduction can be achieved by, for example, one or more of the following: reducing appetite, increasing metabolic rate, reducing fat intake and reducing carbohydrate craving.

Over weight patients include those having a body weight about 10% or more, 20% or more, 30% or more, or 50% or more, than the upper end of a “normal” weight range or Body Mass Index (“BMI”). “Normal” weight ranges are well known in the art and take into account factors such as a patient age, height, and body type.

BMI measures your height/weight ratio. It is determined by calculating weight in kilograms divided by the square of height in meters. The BMI “normal” range is 19-22.

MCH-1R modulating compounds can be provided in a kit. Such a kit typically contains an active compound in dosage forms for administration. A dosage form contains a sufficient amount of active compound such that a beneficial effect can be obtained when administered to a patient during regular intervals, such as 1 to 6 times a day, during the course of 1 or more days. Preferably, a kit contains instructions indicating the use of the dosage form for weight reduction (e.g., to treat obesity or overweight) or stress reduction, and the amount of dosage form to be taken over a specified time period.

Dosing for Therapeutic Applications

Guidelines for pharmaceutical administration in general are provided in, for example, Remington's Pharmaceutical Sciences 18^{th} Edition, Ed. Gennaro, Mack Publishing, 1990, and Modern Pharmaceutics 2^{nd} Edition, Eds. Banker and Rhodes, Marcel Dekker, Inc., 1990, both of which are hereby incorporated by reference herein.

MCH-1R active compounds having appropriate functional groups can be prepared as acid or base salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

MCH-1R active compounds can be administered using different routes including oral, nasal, by injection, and transmucosally. Active ingredients to be administered orally as a suspension can be prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants.

When administered by nasal aerosol or inhalation, compositions can be prepared according to techniques well known in the art of pharmaceutical formulation. Such techniques can involve preparing solutions in saline, employing benzyl alcohol or other suitable preservatives, absorption promoters to enhance bioavailability, fluorocarbons, or other solubilizing or dispersing agents.

Routes of administration include intravenous (both bolus and infusion), intraperitoneal, subcutaneous, topical with or without occlusion, and intramuscular. Injectable solutions or suspensions known in the art include suitable non-toxic, parenterally-acceptable diluents or solvents, such as mannitol, 1,3-butanediol, water, Ringer's solution and isotonic sodium chloride solution. Dispersing or wetting and suspending agents, include sterile, bland, fixed oils, such as synthetic mono- or diglycerides; and fatty acids, such as oleic acid.

Rectal administration in the form of suppositories include the use of a suitable non-irritating excipient, such as cocoa butter, synthetic glyceride esters or polyethylene glycols. These excipients are solid at ordinary temperatures, but liquidify and/or dissolve in the rectal cavity to release the drug.

Suitable dosing regimens for therapeutic applications can be designed taking into account factors well known in the art including age, weight, sex and medical condition of the patient; the severity of the condition to be treated; the route of administration; the renal and hepatic function of the patient; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug. The daily dose for a patient is expected to be between 0.01 and 1,000 mg per adult patient per day.

### EXAMPLES

Examples are provided below to further illustrate different features of the present invention. The examples also illustrate useful methodology for practicing the invention. These examples do not limit the claimed invention.

### Example 1

### Construction of MCH-1R(R141H), MCH-1R(R155A). MCH-1R(Δ316/EGFP)

MCH-1R antagonist binding proteins were created by altering human MCH-1R. Alterations were generated using the QuikChange site-directed mutagenesis kit (Stratagene, La Jolla, Calif.) according to the manufacturers protocol. In brief: A template plasmid is denatured and mutant oligo-primers are annealed. Subsequently, using the non-strand-displacing action of PfuTurbo DNA polymerase primers are extended and incorporated in nicked circular strands. This step is repeated by thermal cycling. At the end of the reaction digestion of the methylated non-mutated parental DNA template is achieved by DpnI followed by transformation of the circular nicked DNA into supercompetent XL-1 Blue E. coli cells which repairs and amplifies the mutant plasmid.

The following combinations of mutant primers were used:

| | |
|---|---|
| R141H+: | |
| 5′-CCATGGCCATTGACCACTACCTGGCCACT | (SEQ. ID. NO. 9) |
| GTCC-3′ | |
| R141H−: | |
| 5′-GGACAGTGGCCAGGTAGTGGTCAATGGCC | (SEQ. ID. NO. 10) |
| ATGG-3′ | |
| R155A+: | |
| 5′-CTCTTCCACGAAGTTCGCGAAGCCCTCTG | (SEQ. ID. NO. 11) |
| TGGCC-3′ | |
| R155A−: | |
| 5′-GGCCACAGAGGGCTTCGCGAACTTCGTGG | (SEQ. ID. NO. 12) |
| AAGAG-3′ | |
| Δ316/EGFP+: | |
| 5′-TTTGTGTACATCGTGCTCTGTGAGGTCGA | (SEQ. ID NO. 13) |
| CGGTACCGCGGGCCCGGG-3′ | |
| Δ316/EGFP−: | |
| 5′-CCCGGGCCCGCGGTACCGTCGACCTCACA | (SEQ. ID. NO. 14) |
| GAGCACGATGTACACAAA-3′ | |

The following templates were used:
MCH-1R(R141H): pcDNA3/MCH-1R
MCH-1R(R155A): pcDNA3/MCH-1R
MCH-1R(Δ316/EGFP): pEGFP-N3-MCH-1R

### Example 2

### Construction of MCH-1R(i2/MC4R)

MCH-1R(i2/MC4R) was created by PCR-based mutagenesis. The resulting MCH-1R antagonist binding protein contains the following amino acid sequence between TM3 and TM4: . . . DRYTFIFYALQYHNIMTVKRATLVICL (SEQ. ID NO. 15) . . . (the underlined sequence is the new sequence inserted in place of the original MCH-1R sequence.)

### Example 3

### Analysis of Radioligand Binding

Membrane binding assays were performed using membrane preparations from transiently transfected HEK293-AEQ17 cells. HEK293-AEQ17 cells (3-5×10⁶ cells were plated in a T75 flask the day before transfection) were transiently transfected with plasmid DNA using LipofectAmine 2000 (Gibco BRL, Rockville, Md.) according to the manufacturer's instructions. After two days membranes were prepared by hypotonic lysis, frozen in liquid nitrogen, and stored at −80° C.

A scintillation proximity assay (SPA) was used to measure the specific binding of [¹²⁵I]Phe¹³Tyr¹⁹-MCH (˜2000 Ci/mmol; NEN Life Sciences, Boston, Mass.) to receptor containing membranes. SPA was carried out using wheat-germ agglutinin-polyvinyltoluene beads (Amersham Corp., Arlington Heights, Ill.), in 96-well OptiPlates (Packard, Meriden, Conn.). Each well contained 0.25 mg of SPA beads, 2-4 μg of membrane protein, and 200 μl of binding buffer. Binding buffer contained 50 mM Tris pH 7.4, 8 mM MgCl₂, 12% glycerol, 0.1% BSA (Sigma, St. Louis, Mo.) and protease inhibitors [4 μg/ml of leupeptin (Sigma, St. Louis, Mo.), 40 μg/ml of Bacitracin (Sigma, St. Louis, Mo.), 5 μg/ml of Aprotinin (Roche Molecular Biochem., Indianapolis, Ind.), and 100 μM AEBSF (Roche Molecular Biochem., Indianapolis, Ind.)].

Assays were optimized with respect to membrane preparations: for HEK293-AEQ17/MCH-1R membranes, 1 μg of membranes per well yielded a >6×specific binding window. Specific binding is defined as the difference between total binding and non-specific binding conducted in the presence of 500 nM unlabeled MCH. Beads were coated with membranes for 20 minutes and dispensed to the 96 wells, various concentrations of test compounds in DMSO were added (final DMSO concentration 1% -2%), then 25 nCi of [¹²⁵I]Phe¹³Tyr¹⁹-MCH was added to the wells. After equilibrating at room temperature for 3 hours, the plates were read in a TopCount (Packard, Meriden, Conn.). IC₅₀ calculations were performed using Prism 3.0 (GraphPad Software, San Diego, Calif.).

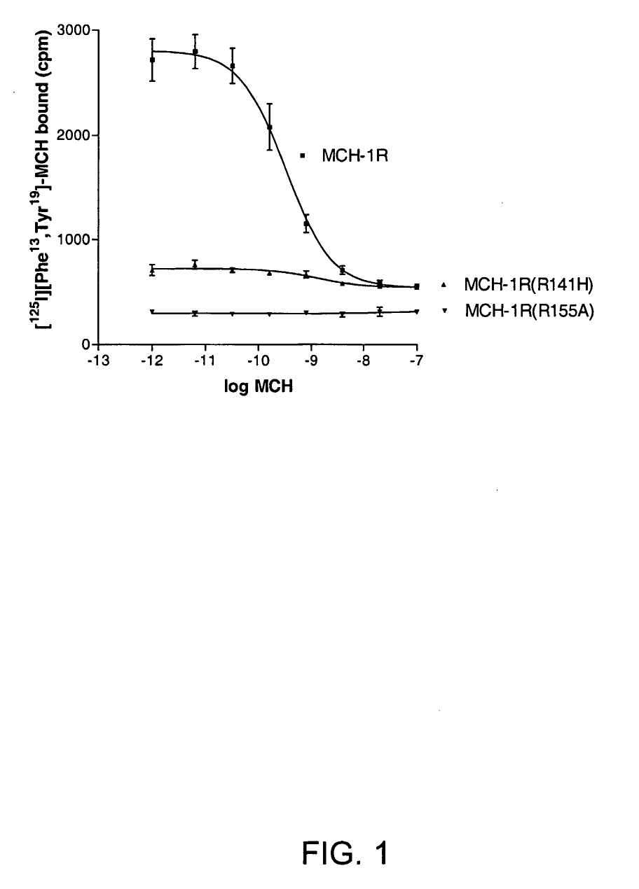

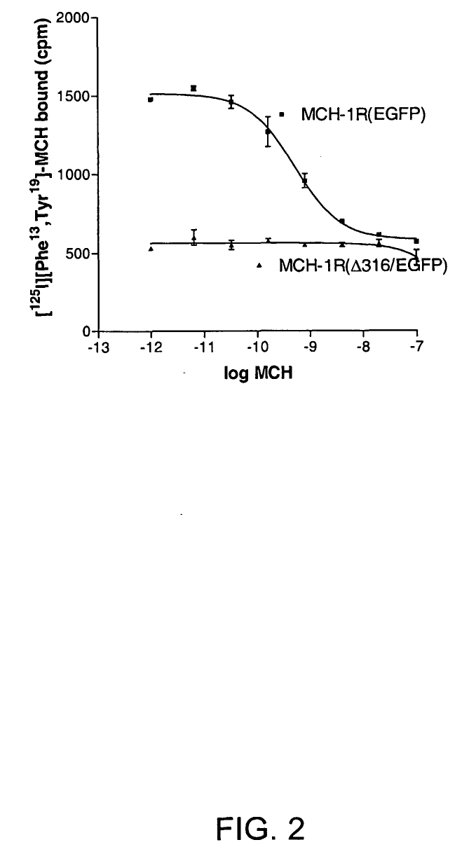

The results of the [¹²⁵I]Phe¹³Tyr¹⁹-MCH agonist binding studies are shown in FIGS. 1 and 2. FIG. 1 illustrates agonist binding to MCH-1R(R141H) and MCH-1R(R155A). FIG. 2 illustrates agonist binding to MCH-1R(Δ316/EGFP).

### Example 4

### Functional Activation Analysis

Functional activation of MCH-1R antagonist binding protein was measured in an aequorin assay. Proteins were introduced in the stable reporter cell line HEK293-AEQ17 in which mobilization of intracellular calcium can be detected by bioluminescence of jelly fish aequorin upon calcium binding.

Bioluminescence was detected using a Luminoskan RT luminometer (Labsystems Inc., Gaithersburg, Md.). HEK293-AEQ17 cells were maintained in D-MEM/high glucose medium (Life Technologies, Rockville, Md.) supplemented with 10% fetal bovine serum, 500 mg/ml G418, 25 mM Hepes at 37° C. with 5% CO₂ in a humidified atmosphere.

HEK293-AEQ17 cells (3-5×10⁶ cells were plated in a T75 flask the day before transfection) were transiently transfected with MCH-1R antagonist binding protein plasmids using LipofectAmine 2000 (Gibco BRL, Rockville, Md.) according to the manufacturer's instructions. After two days cells were incubated with the essential chromophore coelenterazine cp (10 μM; Molecular Probes, Eugene, Oreg.) under reducing conditions (300 μM reduced glutathione in ECB buffer: 140 mM NaCl, 20 mM KCl, 20 mM HEPES-NaOH pH 7.4, 5 mM glucose, 1 mM MgCl₂, 1 mM CaCl₂, 0.1 mg/ml BSA) to charge the apo-aequorin.

The cells were harvested, washed once in ECB medium and resuspended to 500,000 cells/ml. 100 ml of cell suspension (corresponding to 5×10⁴ cells) was then injected into a 96-well test plate, and the integrated light emission was recorded over 30 seconds, in 0.5 second units. 20 μL of lysis buffer (0.1% final Triton X-100 concentration) was then injected and the integrated light emission recorded over 10 seconds, in 0.5 second units. The “fractional response” values for each well were calculated by taking the ratio of the integrated response to the initial challenge to the total integrated luminescence including the Triton X-100 lysis response.

### Example 5

### Antagonist Binding

The ability of MCH-1R antagonist binding protein to bind an MCH-1R antagonist can be evaluated using standard techniques and techniques described herein. For example, the techniques described in Example 3 supra. can modified so that a labeled antagonist is employed.

Examples of MCH-1R antagonists are provided in U.S. Ser. No. 60/310,928 (Attorney Docket NO. 20894PV), filed Aug. 8, 2001, hereby incorporated by reference herein. Peptide antagonists include compounds having the structures (“*” indicates cyclization (S-S)):

| | |
|---|---|
| * * | |
| Ac-Gva-Cys-Met-Leu-Gly-Arg-Val-Tyr-Ava-Ava-Cys-NH₂; | (SEQ. ID. NO. 16) |
| and | |
| * * | |
| Ac-Gva-Cys-Met-Leu-D-Nle-Arg-Val-Tyr-Ava-Ava-Cys-NH₂. | (SEQ. ID. NO. 17) |

“Gva” refers to des-amino-arginine (also known as 5-guanidino-valeric acid). “Ava” refers to 5-aminovaleric acid. D-Nle refers to D-norleucine.

### Example 6

### Sequence Information

Sequences for SEQ. ID. NO. 1-8 are provided below:

MCH-1R(R141H) (SEQ. ID. NO. 1):
MDLEASLLPTGPNASNTSDGPDNLTSAGSPPRTGSISYINIIMPSVFGTI
CLLGIIGNSTVIFAVVKKSKLHWCNNVPDIFIINLSVVDLLFLLGMPFMI
HQLMGNGVWHFGETMCTLITAMDANSQFTSTYILTAMALDHYLATVHPIS
STKFRKPSVATLVICLLWALSFISITPVWLYARLIPFPGGAVGCGIRLPN
PDTDLYWFTLYQFFLAFALPFVVITAAYVRILQRMTSSVAPASQRSLRLR
TKRVTRTAIAICLVFFVCWAPYYVLQLTQLSISRPTLTFVYLYNAAISLG
YANSCLNPFVYIVLCETFRKRLVLSVKPAAQGQLRAVSNAQTADEERTES
KGT
MCH-1R(R155A) (SEQ. ID. NO. 2):
MDLEASLLPTGPNASNTSDGPDNLTSAGSPPRTGSISYINIIMPSVFGTI
CLLGIIGNSTVIFAVVKKSKLHWCNNVPDIFIINLSVVDLLFLLGMPFMI
HQLMGNGVWHFGETMCTLITAMDANSQFTSTYILTAMAIDRYLATVHPIS
STKFAKPSVATLVICLLWALSFISITPVWLYARLIPFPGGAVGCGIRLPN
PDTDLYWFTLYQFFLAFALPFVVITAAYVRILQRMTSSVAPASQRSIRLR
TKRVTRTAIAICLVEFVCWAPYYVLQLTQLSISRPTLTFVYLYNAAISLG
YANSCLNPFVYIYLCETFRKRLVLSVKPAAQGQLRAVSNAQTADEERTES
KGT
MCH-1R(i2/MC4R) (SEQ. ID. NO.3)
MDLEASLLPTGPNASNTSDGPDNLTSAGSPPRTGSISYINIIMPSVFGTI
CLLGIIGNSTVIFAVVKKSKLHWCNNVPDIFIINLSVVDLLFLLGMPFMI
HQLMGNGVWHFGETMCTLITAMDANSQFTSTYILTAMAIDRY [symbol] [symbol]
ATLVICLLWALSFISITPVWLYARLIPFPGGAVGCGIRLPN
PDTDLYWFTLYQFFLAFALPFVVITAAYVRILQRMTSSVAPASQRSIRLR
TKRVTRTAIAICLVFFVCWAPYYVLQLTQLSISRPTLTFVYLYNAAISLG
YANSCLNPFVYIVLCETFRKRLVLSVKPAAQGQLRAVSNAQTADEERTES
KGT
MCH-1R(Δ316/EGFP) (SEQ. ID. NO. 4)
MDLEASLLPTGPNASNTSDGPDNLTSAGSPPRTGSISYINIIMPSVFGTI
CLLGIIGNSTVIFAVVKKSKLHWCNNVPDIFIINLSVVDLLFLLGMPFMI
HQLMGNGVWHFGETMCTLITAMDANSQFTSTYILTAMAIDRYLATVHPIS
STKFRKPSVATLVICLLWALSFISITPVWLYARLIPFPGGAVGCGIRLPN
PDTDLYWFTLYQFFLAFALPFVVITAAYVRILQRMTSSVAPASQRSIRLR
TKRVTRTAIAICLVFFVCWAPYYVLQLTQLSISRPTLTFVYLYNAAISLG
YANSCLNPFVYIVLCEVDGTAGPGSIATMVSKGEELFTGVVPILVELDGD
VNGHKFSVSGEGEGDATYGKLTLKFICTTGKLPVPWPTLVTTLTYGVQCF
SRYPDHMKQHDFFKSAMPEGYVQERTIFFKDDGNYKTRAEVKFEGDTLVN
RIELKGIDFKEDGNILGHKLEYNYNSHNVYIMADKQKNGIKVNFKIRHNI
EDGSVQLADHYQQNTPIGDGPVLLPDNHYLSTQSALSKDPNEKRDHMVLL
EFVTAAGITLGMDELYK
MCH-1R(R141H) (SEQ. ID. NO. 5): Start and stop
codons as well as mutant nucleotide are
highlighted.
ATGGACCTGGAAGCCTCGCTGCTGCCCACTGGTCCCAATGCCAGCAACAC
CTCTGATGGCCCCGATAACCTCACTTCGGCAGGATCACCTCCTCGCACGG
GGAGCATCTCCTACATCAACATCATCATGCCTTCGGTGTTCGGCACCATC
TGCCTCCTGGGCATCATCGGGAACTCCACGGTCATCTTCGCGGTCGTGAA
GAAGTCCAAGCTGCACTGGTGCAACAACGTCCCCGACATCTTCATCATCA
ACCTCTCGGTAGTAGATCTCCTCTTTCTCCTGGGCATGCCCTTCATGATC
CACCAGCTCATGGGCAATGGGGTGTGGCACTTTGGGGAGACCATGTGCAC
CCTCATCACGGCCATGGATGCCAATAGTCAGTTCACCAGCACCTACATCC
TGACCGCCATGGCCATTGACCACTACCTGGCCACTGTCCACCCCATCTCT
TCCACGAAGTTCCGGAAGCCCTCTGTGGCCACCCTGGTGATCTGCCTCCT
GTGGGCCCTCTCCTTCATCAGCATCACCCCTGTGTGGCTGTATGCCAGAC
TCATCCCCTTCCCAGGAGGTGCAGTGGGCTGCGGCATACGCCTGCCCAAC
CCAGACACTGACCTCTACTGGTTCACCCTGTACCAGTTTTTCCTGGCCTT
TGCCCTGCCTTTTGTGGTCATCACAGCCGCATACGTGAGGATCCTGCAGC
GCATGACGTCCTCAGTGGCCCCCGCCTCCCAGCGCAGCATCCGGCTGCGG
ACAAAGAGGGTGACCCGCACAGCCATCGCCATCTGTCTGGTCTTCTTTGT
GTGCTGGGCACCCTACTATGTGCTACAGCTGACCCAGTTGTCCATCAGCC
GCCCGACCCTCACCTTTGTCTACTTATACAATGCGGCCATCAGCTTGGGC
TATGCCAACAGCTGCCTCAACCCCTTTGTGTACATCGTGCTCTGTGAGAC
TTCCGCAAACGCTTGGTCCTGTCGGTGAAGCCTGCAGCCCAGGGGCAGCT
TCGCGCTGTCAGCAACGCTCAGACGGCTGACGAGGAGAGGACAGAAAGCA
AAGGCACCTGATAC
MCH-1R(R141A) (SEQ. ID. NO. 6): Start and stop
codons as well as mutant nucleotide are
highlighted. Nucleic acid sequence (start and stop
codons as well as mutant nucleotide are
highlighted.
ATGGACCTGGAAGCCTCGCTGCTGCCCACTGGTCCCAATGCCAGCAACAC
CTCTGATGGCCCCGATAACCTCACTTCGGCAGGATCACCTCCTCGCACGG
GGAGCATCTCCTACATCAACATCATCATGCCTTCGGTGTTCGGCACCATC
TGCCTCCTGGGCATCATCGGGAACTCCACGGTCATCTTCGCGGTCGTGAA
GAAGTCCAAGCTGCACTGGTGCAACAACGTCCCCGACATCTTCATCATCA
ACCTCTCGGTAGTAGATCTCCTCTTTCTCCTGGGCATGCCCTTCATGATC
CACCAGCTCATGGGCAATGGGGTGTGGCACTTTGGGGAGACCATGTGCAC
CCTCATCACGGCCATGGATGCCAATAGTCAGTTCACCAGCACCTACATCC
TGACCGCCATGGCCATTGACCGCTACCTGGCCACTGTCCACCCCATCTCT
TCCACGAAGTTCGCGAAGCCCTCTGTGGCCACCCTGGTGATCTGCCTCCT
GTGGGCCCTCTCCTTCATCAGCATCACCCCTGTGTGGCTGTATGCCAGAC
TCATCCCCTTCCCAGGAGGTGCAGTGGGCTGCGGCATACGCCTGCCCAAC
CCAGACACTGACCTCTACTGGTTCACCGTGTACCAGTTTTTCCTGGCCTT
TGCCCTGCCTTTTGTGGTCATCACAGCCGCATACGTGAGGATCCTGCAGC
GCATGACGTCCTCAGTGGCCCCCGCCTCCCAGCGCAGCATCCGGCTGCGG
ACAAAGAGGGTGACCCGCACAGCCATCGCCATCTGTCTGGTCTTCTTTGT
GTGCTGGGCACCCTACTATGTGCTACAGCTGACCCAGTTGTCCATCAGCC
GCCCGACCCTCACCTTTGTCTACTTATACAATGCGGCCATCAGCTTGGGC
TATGCCAACAGCTGCCTCAACCCCTTTGTGTACATGGTGCTCTGTGAGAC
GTTCCGCAAACGCTTGGTCCTGTCGGTGAAGCCTGCAGCCCAGGGGCAGC
TTCGCGCTGTCAGCAACGCTCAGACGGCTGACGAGGAGAGGACAGAAAGC
AAAGGCACCTGATAC
MCH-1R(i2/MC4R) (SEQ. ID. NO. 7): Start and stop
codons as well as mutant nucleotide are
highlighted.
ATGGACCTGGAAGCCTCGCTGCTGCCCACTGGTCCCAATGCCAGCAACAC
CTCTGATGGCCCCGATAACCTCACTTCGGCAGGATCACCTCCTCGCACGG
GGAGCATCTCCTACATCAACATCATCATGCCTTCGGTGTTCGGCACCATC
TGCCTCCTGGGCATCATCGGGAACTCCACGGTCATCTTCGCGGTCGTGAA
GAAGTCCAAGCTGCACTGGTGGAACAACGTCCCCGACATCTTCATCATCA
ACCTCTCGGTAGTAGATCTCCTCTTTCTCCTGGGCATGCCCTTCATGATC
CACCAGCTCATGGGCAATGGGGTGTGGCACTTTGGGGAGACCATGTGCAC
CCTCATCACGGCCATGGATGCCAATAGTCAGTTCACCAGCACCTACATCC
TGACCGCCATGGCCATTGACCGCTAC [symbol] [symbol]
GCGGGCCACCCTGGTGATCTGCCTCCT
GTGGGCCCTCTCCTTCATCAGCATCACCCCTGTGTGGGTGTATGCCAGAC
TCATCCCCTTCCCAGGAGGTGCAGTGGGCTGCGGCATACGCCTGCCCAAC
CCAGACACTGACCTCTACTGGTTCACCCTGTACCAGTTTTTCCTGGCCTT
TGCCCTGCCTTTTGTGGTCATCACAGCCGCATACGTGAGGATCCTGCAGC
GCATGACGTCCTCAGTGGCCCCCGCCTCCCAGCGCAGCATCCGGCTGCGG
ACAAAGAGGGTGACCCGCACAGCCATCGCCATCTGTCTGGTCTTCTTTGT
GTGCTGGGCACCCTACTATGTGCTACAGCTGACCCAGTTGTCCATCAGCC
GCCCGACCCTCACCTTTGTCTACTTATACAATGCGGCCATCAGCTTGGGC
TATGCCAACAGCTGCCTCAACCCCTTTGTGTACATCGTGCTCTGTGAGAC
GTTCCGCAAACGCTTGGTCCTGTCGGTGAAGCCTGCAGCCCAGGGGCAGC
TTCGCGCTGTCAGCAACGCTCAGACGGCTGACGAGGAGAGGACAGAAAGC
AAAGGCACCTGATAC
MCH-1R(Δ316/EGFP) (SEQ. ID. NO. 8): Start codon
and start and stop codons for MCH-1R and EGFP,
respectively, are highlighted. A 12 amino acid
linker sequence is denoted in lower case.
ATGGACCTGGAAGCCTCGCTGCTGCCCACTGGTCCCAATGCCAGCAACAC
CTCTGATGGCCCCGATAACCTCACTTCGGCAGGATCACCTCCTCGCACGG
GGAGCATCTCCTACATCAACATCATCATGCCTTCGGTGTTCGGCACCATC
TGCCTCCTGGGCATCATCGGGAACTCCACGGTCATCTTCGCGGTCGTGAA
GAAGTCCAAGCTGCACTGGTGCAACAACGTCCCCGACATCTTCATCATCA
ACCTCTCGGTAGTAGATCTCCTCTTTCTCCTGGGCATGCCCTTCATGATC
CACCAGCTCATGGGCAATGGGGTGTGGCACTTTGGGGAGACCATGTGCAC
CCTCATCACGGCCATGGATGCCAATAGTCAGTTCACCAGCACCTACATCC
TGACCGCCATGGCCATTGACCGCTACCTGGCCACTGTCCACCCCATCTCT
TCCACGAAGTTCCGGAAGCCCTCTGTGGCCACCCTGGTGATCTGCCTCCT
GTGGGCCCTCTCCTTCATCAGCATCACCCCTGTGTGGCTGTATGCCAGAC
TCATCCCCTTCCCAGGAGGTGCAGTGGGCTGCGGCATACGCCTGCCCAAC
CCAGACACTGACCTCTACTGGTTCACCCTGTACCAGTTTTTCCTGGCCTT
TGCCCTGCCTTTTGTGGTCATCACAGCCGCATACGTGAGGATCCTGCAGC
GCATGACGTCCTCAGTGGCCCCCGCCTCCCAGCGCAGCATCCGGCTGCGG
ACAAAGAGGGTGACCCGCACAGCCATCGCCATCTGTCTGGTCTTCTTTGT
GTGCTGGGCACCCTACTATGTGCTACAGCTGACCCAGTTGTCCATCAGCC
GCCCGACCCTCACCTTTGTCTACTTATACAATGCGGCCATCAGCTTGGGC
TATGCCAACAGCTGCCTCAACCCCTTTGTGTACATCGTGCTCTGTGAGgt
cgacggtaccgcgggcccgggatccatcgccaccATGGTG
AGCAAGGGCG AGGAGCTGTT CACCGGGGTG GTGCCCATCC
TGGTCGAGCT GGACGGCGAC GTAAACGGCC ACAAGTTCAG
CGTGTCCGGC GAGGGCGAGG GCGATGCCAC CTACGGCAAG
CTGACCCTGA AGTTCATCTG CACCACCGGC AAGCTGCCCG
TGCCCTGGCC CACCCTCGTG ACCACCCTGA CCTACGGCGT
GCAGTGCTTC AGCCGCTACC CCGACCACAT GAAGCAGCAC
GACTTCTTCA AGTCCGCCAT GCCCGAAGGC TACGTCCAGG
AGCGCACCAT CTTCTTCAAG GACGACGGCA ACTACAAGAC
CCGCGCCGAG GTGAAGTTCG AGGGCGACAC CCTGGTGAAC
CGCATCGAGC TGAAGGGCAT CGACTTCAAG GAGGACGGCA
ACATCCTGGG GCACAAGCTG GAGTACAACT ACAACAGCCA
CAACGTCTAT ATCATGGCCG ACAAGCAGAA GAACGGCATC
AAGGTGAACT TCAAGATCCG CCACAACATC GAGGACGGCA
GCGTGCAGCT CGCCGACCAC TACCACGAGA ACACCCCCAT
CGGCGACGGC CCCGTGCTGC TGCCCGACAA CCACTACCTG
AGCACCCAGT CCGCCCTGAG CAAAGACCCC AACGAGAAGC
GCGATCACAT GGTCCTGCTG GAGTTCGTGA CCGCCGCCGG
GATCACTCTC GGCATGGACG AGCTGTACAA GTAA

Other embodiments are within the following claims. While several embodiments have been shown and described, various modifications may be made without departing from the spirit and scope of the present invention.

1. A melanin-concentrating hormone receptor type 1 (MCH-1R) antagonist binding protein selected from the group consisting of:
a) a first MCH-1R antagonist binding protein, wherein said first MCH-1R antagonist binding protein has one or more alterations in the second intracelullar loop region that render MCH-1 R substantially inactive to agonist activation; and
b) a second MCH-1R antagonist binding protein, wherein said second MCH-1R antagonist binding protein has one or more alterations in the C-terminal region that render MCH-1R substantially inactive to agonist activation.

2. The MCH-1R antagonist binding protein of claim 1, wherein said MCH-1 R antagonist binding protein is said first MCH-1 R antagonist binding protein.

3. The MCH-1R antagonist binding protein of claim 2, wherein said MCH-1R antagonist binding protein consists of the amino acid sequence of SEQ ID NO: 1.

4. The MCH-1R antagonist binding protein of claim 2, wherein said MCH-1R antagonist binding protein consists of the amino acid sequence of SEQ ID NO: 2.

5. The MCH-1R antagonist binding protein of claim 2, wherein said MCH-1R antagonist binding protein consists of the amino acid sequence of SEQ ID NO: 3.

6. The MCH-1R antagonist binding protein of claim 1, wherein said MCH-1R antagonist binding protein is said second MCH-1R antagonist binding protein.

7. The MCH-1R antagonist binding protein of claim 6, wherein said MCH-1R antagonist binding protein consists of the amino acid sequence of SEQ ID NO: 4.

8. A nucleic acid comprising a nucleotide sequence encoding the MCH-1R antagonist binding protein claim 1.

9. A nucleic acid comprising a nucleotide sequence selected from the group consisting of: SEQ ID NO: 5, SEQ ID NO: 6., SEQ ID NO: 7, and SEQ ID NO: 8.

10. The nucleic acid of claim 9, wherein said nucleotide sequence consists of SEQ ID NO: 5.

11. The nucleic acid of claim 9, wherein said nucleotide sequence consists of SEQ ID NO: 6.

12. The nucleic acid of claim 9, wherein said nucleotide sequence consists of SEQ ID NO: 7.

13. The nucleic acid of claim 9, wherein said nucleotide sequence consists of SEQ ID NO: 8.

14. The nucleic acid claim 8, wherein said nucleic acid is an expression vector.

15. A recombinant cell comprising the expression vector of claim 14, wherein said nucleotide sequence is functionally coupled to a promoter recognized by said cell.

16. A method of screening for a compound able to bind MCH-1R comprising the steps of:
a) contacting the MCH-1R antagonist binding protein of claim 1 with a said compound; and
b) measuring the ability of said compound to bind to said MCH-1 R antagonist binding protein.

17. A method of preparing a MCH-1R antagonist binding protein comprising the step of growing the recombinant cell of claim 15 under conditions wherein said protein is expressed from said expression vector.
